(19) United States
(12) Patent Application Publication
(10) Pub. No.: US 2009/0118786 A1
(43) Pub. Date: May 7, 2009

(54) AUTOMATED FITTING SYSTEM FOR DEEP BRAIN STIMULATION

(75) Inventors: Paul Milton Meadows, Glendale, CA (US); Michael Adam Moffitt, Valencia, CA (US)

Correspondence Address: Vista IP Law Group LLP, 2040 MAIN STREET, 9TH FLOOR, IRVINE, CA, 92614, US

(73) Assignee: ADVANCED BIONICS CORPORATION, Valencia, CA (US)

(21) Appl. No.: 11/934,731

(22) Filed: Nov. 2, 2007

(51) Int. Cl. A61N 1/05

(52) U.S. Cl. 607/45

(57) ABSTRACT

Methods, systems, and external programmers provide therapy to a patient having a dysfunction. In one aspect, stimulation energy is conveyed from a neurostimulator to electrodes located within a tissue region of the patient, thereby changing the status of the dysfunction. A physiological end-function of the patient indicative of the changed status of the dysfunction is measured, and stimulation parameters are programmed into the neurostimulator based on the measured physiological end-function. In another aspect, electrodes are placed adjacent to a tissue region of the patient, and stimulation energy is conveyed from the electrodes to the tissue region in accordance with the stimulation parameters, thereby changing the status of the dysfunction. A physiological end-function of the patient indicative of the changed status of the dysfunction is measured, and the stimulation parameters are adjusted based on the measured physiological end-function.

### FIELD OF THE INVENTION

The present inventions relate to the treatment of movement disorders, and more particularly, to deep brain stimulation (DBS) systems and methods.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications, such as angina pectoris and incontinence. Further, in recent investigations, Peripheral Nerve Stimulation (PNS) systems have demonstrated efficacy in the treatment of chronic pain syndromes and incontinence, and a number of additional applications are currently under investigation. More pertinent to the present inventions described herein, Deep Brain Stimulation (DBS) has been applied therapeutically for well over a decade for the treatment of neurological disorders, including Parkinson's Disease, essential tremor, dystonia, and epilepsy, to name but a few. Further details discussing the treatment of diseases using DBS are disclosed in U.S. Pat. Nos. 6,845,267, 6,845,267, and 6,950,707, which are expressly incorporated herein by reference.

Each of these implantable neurostimulation systems typically includes one or more electrode carrying stimulation leads, which are implanted at the desired stimulation site, and a neurostimulator implanted remotely from the stimulation site, but coupled either directly to the stimulation lead(s) or indirectly to the stimulation lead(s) via a lead extension. The neurostimulation system may further comprise a handheld remote control (RC) to remotely instruct the neurostimulator to generate electrical stimulation pulses in accordance with selected stimulation parameters. The RC may, itself, be programmed by a technician attending the patient, for example, by using a Clinician's Programmer (CP), which typically includes a general purpose computer, such as a laptop, with a programming software package installed thereon.

Thus, in accordance with the stimulation parameters programmed by the RC and/or CP, electrical pulses can be delivered from the neurostimulator to the stimulation electrode(s) to stimulate or activate a volume of tissue in accordance with a set of stimulation parameters and provide the desired efficacious therapy to the patient. The best stimulus parameter set will typically be one that delivers stimulation energy to the volume of tissue that must be stimulated in order to provide the therapeutic benefit (e.g., treatment of movement disorders), while minimizing the volume of non-target tissue that is stimulated. A typical stimulation parameter set may include the electrodes that are acting as anodes or cathodes, as well as the amplitude, duration, and rate of the stimulation pulses.

When a neurostimulation system is implanted within a patient, a fitting procedure is typically performed to ensure that the stimulation leads and/or electrodes are properly implanted in effective locations of the patient, as well as to select one or more effective sets of stimulation parameters for the patient. In some electrical stimulation treatments, the fitting procedure may be effectively directed in response to patient feedback. For example, in SCS for providing pain relief, patients can feel the effects of the stimulation pulses and the change in their pain status, and thus, may provide verbal feedback as to the efficacy of the stimulation, and thus, the proper location of the stimulation leads and/or electrodes and the stimulation parameters to be used in delivering the electrical pulses to the patient on a long-term basis.

Unlike with SCS, patients receiving DBS cannot feel the effects of stimulation, and the effects of the stimulation may be difficult to observe, are typically subjective, or otherwise may take a long time to become apparent. This makes it difficult to set the stimulation parameters appropriately or otherwise select stimulation parameters that result in optimal treatment for the patient and/or optimal use of the stimulation resources. Significantly, non-optimal electrode placement and stimulation parameter selections may result in excessive energy consumption due to stimulation that is set at too high an amplitude, too wide a pulse width, or too fast a frequency; inadequate or marginalized treatment due to stimulation that is set at too low an amplitude, too narrow a pulse width, or too slow a frequency; or stimulation of neighboring cell populations that may result in undesirable side effects. All of these issues are poorly addressed by the present-day DBS fitting techniques. In addition, after the DBS system has been implanted and fitted, the patient may have to schedule another visit to the physician in order to adjust the stimulation parameters of the DBS system if the treatment provided by the implanted DBS system is no longer effective or otherwise is not therapeutically or operationally optimum due to, e.g., disease progression, motor re-learning, or other changes.

While DBS systems have been disclosed that utilize a closed-loop method that involves sensing electrical signals within the brain of the patient and automatically adjusting the electrical stimulation delivered to a target region within the brain of the patient (see, e.g., U.S. Pat. No. 5,683,422), such a system requires the implantation of an additional lead within the brain. In addition, the electrical signals sensed within the brain are not easily correlatable to the disorder currently experienced by the patient. Furthermore, such a system is not designed to be used in a fitting procedure, including physical adjustment of the leads and programming of the stimulation parameters.

There, thus, remains a need for a DBS system that can be more easily fitted to a patient in order to optimize treatment of a patient suffering from a disease.

### SUMMARY OF THE INVENTION

A method of providing therapy to a patient having a dysfunction is provided. In one method, the dysfunction is a motor dysfunction (e.g., a gait dysfunction, posture dysfunction, balance dysfunction, motor control dysfunction, speech dysfunction, etc.), and may be caused by neurological disorder, such as Parkinson's Disease, essential tremor, dystonia, epilepsy, etc. The method comprises conveying stimulation energy from a neurostimulator to at least one implanted electrode located within a tissue region of the patient, thereby changing the status of the dysfunction. The tissue region may be located anywhere in the patient's body, but in the preferred method, is located in the brain where motor dysfunctions often originate. The method further comprises measuring a physiological end-function of the patient indicative of the changed status of the dysfunction, and programming at least one stimulation parameter into the neurostimulator based on the measured physiological end-function. The measured physiological end-function may be, e.g., a kinematic function, an electrical muscle impulse, a speech pattern, etc., and the stimulator parameter(s) may be, e.g., a pulse amplitude (including the relative amplitudes of current or voltage through electrodes of like polarity), pulse width, pulse rate, or electrode combination. In one method, the physiological end-function is non-invasively measured.

One method further comprises conveying stimulation energy from the neurostimulator to the tissue region of the patient in accordance with the stimulation parameter(s), thereby improving the status of the dysfunction. Another method further comprises quantifying the dysfunction based on the measured physiological end-function, in which case, the stimulation parameter(s) may be programmed into the neurostimulator based on the quantified dysfunction. Still another method further comprises automatically determining the stimulation parameter(s) in response to the measured physiological end-function. The automatic determination of the stimulation parameter(s) may be performed in any one of a variety manners, e.g., heuristically or by correlating the measured physiological end-function to a predetermined data set. The method may optionally comprise implanting the neurostimulator into the patient.

In accordance with a second aspect of the present inventions, a neurostimulation system is provided. The neurostimulation system comprises at least one electrical terminal, output stimulation circuitry configured for outputting stimulation energy to the electrical terminal(s), control circuitry configured for controlling the stimulation energy output by the output stimulation circuitry, monitoring circuitry configured for measuring a physiological end-function of a patient indicative of a changed status of a dysfunction of a patient, and processing circuitry configured for programming the control circuitry with at least one stimulation parameter based on the measured physiological end-function. The dysfunction, measured physiological end-function, and stimulation parameter(s) may be the same as those described above.

In one embodiment, the monitoring circuitry is configured for non-invasively measuring the physiological end-function. In another embodiment, the processing circuitry is configured for programming the control circuitry with the stimulation parameter(s) to improve the status of the dysfunction when the output stimulation circuitry outputs the stimulation energy to the electrical terminal(s). In still another embodiment, the monitoring circuitry is configured for quantifying the dysfunction based on the measured physiological end-function, in which case, the processing circuitry may be configured for programming the stimulation parameter(s) into the control circuitry based on the quantified dysfunction. In still another embodiment, the processing circuitry is configured for automatically determining the stimulation parameter(s) in response to the measured physiological end-function, e.g., in the manner discussed above. In yet another embodiment, the system further comprises telemetry circuitry configured for wirelessly conveying the stimulation parameter(s) from the processing circuitry to the control circuitry. An optional embodiment may comprise a case containing the electrical terminal(s), output stimulation circuitry, and control circuitry to form a neurostimulator, e.g., an implantable neurostimulator. The monitoring circuitry and the processing circuitry may be contained in one or more computers.

In accordance with a third aspect of the present inventions, an external programmer for a neurostimulator is provided. The external programmer comprises input circuitry configured for receiving information indicative of a changed status of a dysfunction of a patient. The information may be, e.g., a measured physiological end-function or a quantified dysfunction, the details of which are discussed above. The programmer further comprises processing circuitry configured for automatically determining at least one programmable stimulation parameter based on the received information, and output circuitry configured for transmitting the programmable stimulation parameter to the neurostimulator. The programmable stimulation parameter(s) may be the same as those discussed above, and the programmable stimulation parameter(s) may be determined in the same manner described above. In one embodiment, the processing circuitry is configured for defining the programmable stimulation parameter(s), such that the status of the dysfunction is improved when stimulation energy is delivered to the patient in accordance with the programmable stimulation parameter(s). In another embodiment, the output circuitry comprises telemetry circuitry, and the input circuitry, processing circuitry, and output circuitry are contained in a single case.

In accordance with a fourth aspect of the present inventions, a method of providing therapy to a patient having a dysfunction is provided. In one method, the dysfunction is a motor dysfunction (e.g., a gait dysfunction, posture dysfunction, balance dysfunction, motor control dysfunction, speech dysfunction, etc.), and may be caused by neurological disorder, such as Parkinson's Disease, essential tremor, dystonia, epilepsy, etc. The method comprises placing at least one electrode adjacent to a tissue region of the patient, and conveying stimulation energy from the electrode(s) to the tissue region in accordance with at least one stimulation parameter (e.g., a pulse amplitude, pulse width, pulse rate, electrode combination, etc.), thereby changing the status of the dysfunction. The tissue region may be located anywhere in the patient's body, but in the preferred method, is located in the brain where dysfunctions often originate. The method further comprises measuring a physiological end-function of the patient indicative of the changed status of the dysfunction, and automatically adjusting the stimulation parameter(s) based on the measured physiological end-function. The measured physiological end-function may be, e.g., a kinematic function, an electrical muscle impulse, a speech pattern, etc. In one method, the physiological end-function is non-invasively measured.

One method comprises quantifying the dysfunction based on the measured physiological end-function, in which case, the stimulation parameter(s) may be automatically adjusted based on the quantified dysfunction. In another method, the stimulation parameter(s) are automatically adjusted to improve the status of the dysfunction. For example, a value of the stimulation parameter(s) may be adjusted in one direction if the measured physiological end-function indicates an improvement in the status of the dysfunction, and may be adjusted in another direction if the measured physiological end-function indicates a degradation in the status of the dysfunction. Still another method comprises conveying stimulation energy from the electrode(s) to the tissue region in accordance with the adjusted stimulation parameter(s), thereby changing the status of the dysfunction. Yet another method comprises implanting the neurostimulator within the patient, coupling the electrode(s) to the neurostimulator, and programming the neurostimulator with the adjusted stimulation parameter(s).

In accordance with a fifth aspect of the present inventions, a neurostimulation system is provided. The neurostimulation system comprises at least one electrical terminal, output stimulation circuitry configured for outputting stimulation energy to the electrical terminal(s) in accordance with at least one stimulation parameter, monitoring circuitry configured for measuring a physiological end-function of a patient indicative of a changed status of a dysfunction of a patient, and processing circuitry configured for adjusting the stimulation parameter(s) based on the measured physiological end-function. The dysfunction, measured physiological end-function, and stimulation parameter(s) may be the same as those described above.

In one embodiment, the monitoring circuitry is further configured for quantifying the dysfunction based on the measured physiological end-function, in which case, the processing circuitry may be configured for automatically adjusting the stimulation parameter(s) based on the quantified dysfunction. In another embodiment, the processing circuitry is configured for automatically adjusting the stimulation parameter(s) to improve the status of the dysfunction; for example, in the manner described above. In still another embodiment, the system further comprises a stimulation lead carrying at least one electrode electrically coupled to the at least one electrical terminal. In yet another embodiment, the system further comprises telemetry circuitry, in which case, the processing circuitry is configured for wirelessly adjusting the stimulation parameter(s). An optional embodiment may comprise a case containing the electrical terminal(s), output stimulation circuitry, and control circuitry to form a neurostimulator, e.g., an implantable neurostimulator. The monitoring circuitry and the processing circuitry may be contained in one or more computers.

In accordance with a sixth aspect of the present inventions, an external programmer for a neurostimulator is provided. The external programmer comprises input circuitry configured for receiving information indicating a status of a dysfunction of a patient, processing circuitry configured for automatically adjusting at least one stimulation parameter based on the received information, and output circuitry configured for transmitting the adjusted stimulation parameter(s) to the neurostimulator. The received information may be, e.g., a measured physiological end-function or a quantified dysfunction, the details of which are discussed above. The programmable stimulation parameter(s) may be the same as those discussed above, and the programmable stimulation parameter(s) may be determined in the same manner described above. In one embodiment, the processing circuitry is configured for automatically adjusting the at least one stimulation parameter to improve the status of the dysfunction; for example, in the same manner described above. In another embodiment, the output circuitry is telemetry circuitry, and the input circuitry, processing circuitry, and output circuitry are contained in a single case.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of a Deep Brain Stimulation (DBS) system constructed in accordance with one embodiment of the present inventions;

FIG. 2 is a block diagram of the internal components of an implantable pulse generator (IPG) used in the DBS system of FIG. 1;

FIG. 3 is front view of a remote control (RC) used in the DBS system of FIG. 1;

FIG. 4 is a block diagram of the internal components of the RC of FIG. 3;

FIG. 5 is a block diagram of the internal components of a clinician's programmer (CP) used in the DBS system of FIG. 1;

FIG. 6 is a flow diagram illustrating a method of programming the IPG of FIG. 2 using the RC of FIGS. 3 and 4 or the CP of FIG. 5; and

FIG. 7 is a cross-sectional view of a patient's head showing the implantation of stimulation leads and an IPG of the DBS system of FIG. 1.

### DETAILED DESCRIPTION OF THE EMBODIMENTS

At the outset, it is noted that the present invention may be used with an implantable pulse generator (IPG), radio frequency (RF) transmitter, or similar neurostimulator, that may be used as a component of numerous different types of stimulation systems. The description that follows relates to a Deep Brain Stimulation (DBS) system. However, it is to be understood that, while the invention lends itself well to applications in DBS, the invention, in its broadest aspects, may not be so limited. Rather, the invention may be used with any type of implantable electrical circuitry used to stimulate tissue for the treatment of a dysfunction, such as, e.g., a motor dysfunction.

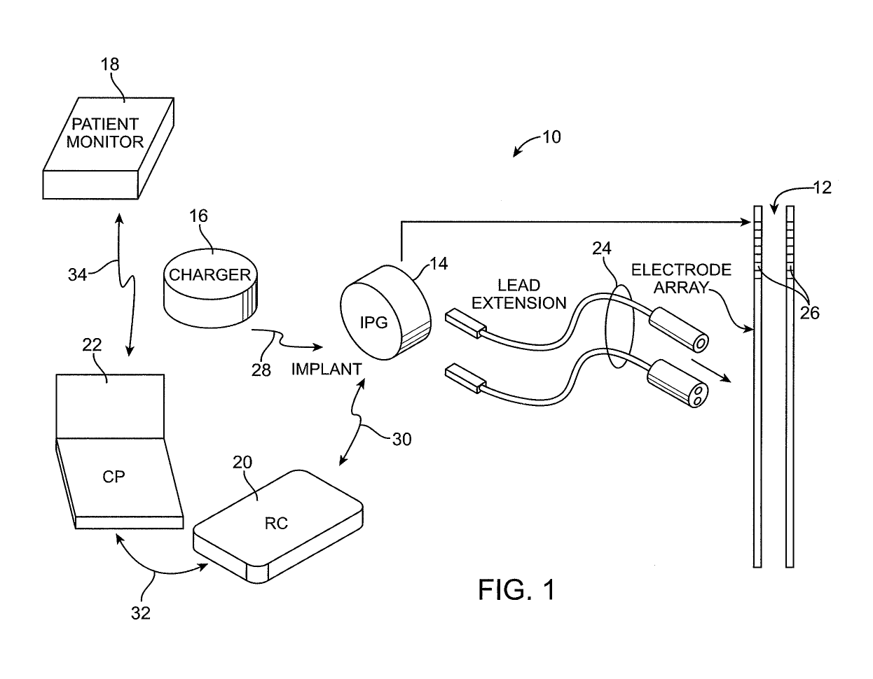

Turning first to FIG. 1, an exemplary DBS system 10 constructed in accordance with one embodiment of the present inventions generally includes one or more (in this case, two) implantable stimulation leads 12, an implantable pulse generator (IPG) 14 (or alternatively RF receiver-stimulator), an external charger 16, a patient monitor 18, an external remote controller (RC) 20, and a clinician's programmer (CP) 24.

The IPG 14 is physically connected via one or more lead extensions 24 to the stimulation leads 12, which carry a plurality of electrodes 26 arranged in an array. In the illustrated embodiment, the electrodes 26 are arranged in-line along the stimulation leads 12. In the illustrated embodiment, each stimulation lead 12 carries eight electrodes 26. Of course, other numbers of electrodes can be carried by each stimulation lead 12, e.g., two, four, six, etc., and any number of stimulation leads 12 can be used, including a single lead. The IPG 14 comprises an outer case for housing the electronic and other components (described in further detail below), and a connector (not shown) in which the proximal end of the lead extension 24 mates with the IPG 14, which then at its distal end has a connector which mates with the stimulation lead 12 mates in a manner that electrically couples the electrodes 26 to the electronics within the outer case. The outer case is composed of an electrically conductive, biocompatible material, such as titanium, and forms a hermetically sealed compartment, wherein the internal electronics are protected from the body tissue and fluids. In some cases, the outer case serves as an electrode, as will be described in further detail below.

As will be described in further detail below, the IPG 14 includes pulse generation circuitry that delivers the electrical stimulation energy to the electrodes 26 in accordance with a set of stimulation parameters. Such stimulation parameters may comprise electrode combinations, which define the electrodes that are activated as anodes (positive), cathodes (negative), and turned off (zero), and electrical pulse parameters, which define the pulse amplitude (measured in milliamps or volts depending on whether the IPG 14 supplies constant current or constant voltage to the electrodes 26), pulse width (measured in microseconds), and pulse rate (measured in pulses per second). Electrical stimulation will occur between two (or more) activated electrodes, one of which may be the IPG case. Simulation energy may be transmitted to the tissue in a monopolar manner; that is, between one of the electrodes 26 and the IPG case, or multipolar manner (e.g., bipolar, tripolar, etc.); that is, between two or more of the electrodes 26.

The external charger 16 is a portable device used to transcutaneously charge the IPG 14 via an inductive link 28. For purposes of brevity, the details of the external charger 24 will not be described herein. Details of exemplary embodiments of external chargers are disclosed in U.S. Pat. No. 6,895,280, which has been previously incorporated herein by reference.

The patient monitor 18 is used to measure a physiological end-function indicative of the changed status of the dysfunction from which the patient suffers. For the purposes of this specification, a physiological end-function is a physiological function that manifests itself outside of the brain. The physiological end-function is preferably measured using a non-invasive means (i.e., without having to create an opening within the patient) or otherwise a means that does not require penetration into the patient's brain. Various non-invasive means for measuring the physiological end-function are described in further detail below. Alternatively, the physiological end-function may be invasively measured. The measured physiological end-function may be, e.g., a kinematic action, an electrical muscle impulse, or a speech pattern. The dysfunction may be a motor dysfunction, e.g., a gait dysfunction, posture dysfunction, balance dysfunction, motor control dysfunction (e.g., spasticity, bradykinesia, rigidity), a speech impediment, etc., which may be caused by any one of a variety of diseases, including Parkinson's Disease, essential tremor, dystonia, and epilepsy. The dysfunction may also be a non-motor dysfunction, e.g., psychological, hormonal, etc. The patient monitor 18 may optionally quantify the dysfunction based on the measured physiological end-function; for example, by assigning a numerical value to the dysfunction (e.g., from 1 to 10, with 1 meaning that the dysfunction is non-existent and 10 meaning that the dysfunction is extreme). As will be described in further detail below, the measured physiological end-function or quantified dysfunction information can be used to adjust the stimulation parameters in accordance with which the stimulation energy is delivered from the IPG 14.

The patient monitor 18 may be physically located in a clinical setting where direct physician/assistant control may be exercised under control conditions, or may be located with the patient at a remote setting to allow more limited and/or gradual adjustment of the stimulation parameters. Thus, the patient monitor 18 can be utilized at any time during the treatment continuum to record pre-implant performance, post-implant performance, and follow-up adjustment opportunities.

The RC 20 may be used to telemetrically control the IPG 14 via a bi-directional RF communications link 30 by transmitting stimulation parameters to the IPG 14 or otherwise adjusting the stimulation parameters stored in the IPG 14. Such control allows the IPG 14 to be turned on or off and to be programmed with different stimulation programs after implantation. Once the IPG 14 has been programmed, and its power source has been charged or otherwise replenished, the IPG 14 may function as programmed without the RC 20 being present.

The CP 22 provides clinician-specified stimulation parameters for programming the IPG 14 in the operating room and in follow-up sessions. The CP 22 may perform this function by communicating with the RC 20 via an IR communications link 32 to indirectly program the IPG 14 with the stimulation parameters. The CP 22 may, at the same time, program the RC 20 with the stimulation parameters, so that the RC 20 can subsequently program or otherwise control the IPG 14 using the stimulation parameters programmed into the RC 20. Alternatively, the CP 22 may directly program the stimulation parameters into the IPG 14 via an RF communications link (not shown) without the aid of the RC 20.

Significantly, the CP 22 may operate in a manual mode or an automated mod. In a manual mode, the CP 22 can be used to program stimulation parameters into the IPG 14 in a conventional manner. In the automated mode, the CP 22 can be used to automatically program stimulation parameters into the IPG 14. In particular, the CP 22 can automatically determine the stimulation parameters to be programmed into the IPG 14 based on the physiological end-function measured by the patient monitor 18. To this end, the CP 22 may receive measured physiological end-function information from the patient monitor 18 via an IR communications link 34. Alternatively, the CP 22 may be coupled to the patient monitor 18 via a cable (not shown). If the patient monitor 18 quantifies the dysfunction based on the measured physiological end-functions, the CP 22 may receive the quantified dysfunction information from the patient monitor 18 via the IR communications link 34, and automatically determine the programmed stimulation parameters based on the quantified dysfunction information. Alternatively, the CP 22, itself, may quantify the dysfunction based on the measured physiological end-function information received from the patient monitor 18. Notably, the CP 22 may automatically determine the stimulation parameters to be programmed into the IPG 14 without user intervention, or may, e.g., provide suggested stimulation parameters, which can be selected by the clinician to ultimately adjust the stimulation parameters programmed into the IPG 14. In any event, the programmed stimulation parameters determined by the CP 22 are intended to improve the status of the dysfunction suffered by the patient.

For example, the CP 22 may control the stimulation energy output by the IPG 14 by adjusting the stimulation parameters in the IPG 14. The patient monitor 18 may measure the physiological end-function of the patient again to determine the effect that the adjustment of the stimulation parameters had on the dysfunction. This process can be repeated until optimized or otherwise effective or improved stimulation parameters are determined, which can then be programmed into the IPG 14. Any delay between the change in the stimulation parameters and the measurement of the physiological end-functions would be controlled and would be affected by the type of dysfunction, physical condition of the patient, the effects of any drugs, etc., allowing the changes in stimulation to take effect before another measurement of physiological end-functions is performed again. Changes due to disease progression, motor re-learning, or other changes that effect the status of the dysfunction can be triggered for re-evaluation of the stimulation parameters programmed into the IPG 14.

The RC 20 can be operated in a manual mode that allows a patient to program stimulation parameters into the IPG 14 in a conventional manner. In alternative embodiments, wherein the patient monitor 18 is located within the patient in a remote setting, the RC 20 may operated in an automated mode in which it automatically determines the stimulation parameters to be programmed into the IPG 14 based on the physiological end-function measured by the patient monitor 18 or the dysfunction quantified by the patient monitor 18, in which case, the RC 20 may be coupled to the patient monitor 18 via an IR communications link (not shown).

The CP 22, or alternatively the RC 20, may determine the improved stimulation parameters based on the measured physiological end-function or quantified dysfunction in any one of a variety of manners to improve the status of the dysfunction. In one embodiment, the stimulation parameters are adjusted using a heuristic approach.

For example, a value of at least one of the stimulation parameters may be incrementally adjusted in one direction (e.g., increasing the pulse amplitude, pulse width, or pulse rate) if the measured physiological end-function indicates an improvement in the status of the dysfunction, and incrementally adjusted in another direction (e.g., decreasing the pulse amplitude, pulse width, or pulse rate) if the measured physiological end-function indicates a degradation in the status of the dysfunction. The value of the stimulation parameters may be incrementally adjusted in the one direction until the measured physiological end-function indicates no further improvement in the status of the dysfunction or until a parameter limit is reached. These stimulation parameters can then be selected as the stimulation parameters to be programmed into the IPG 14.

As another example, different combinations of electrodes may be selected that improve the status of the dysfunction. In one embodiment, the stimulation energy may be gradually steered up or down the leads 12. That is, the stimulation energy may be gradually steered in one direction if the measured physiological end-function indicates an improvement in the status of the dysfunction, and gradually steered in another direction if the measured physiological end-function indicates a degradation in the status of the dysfunction. The improved stimulation parameters, and in this case, the electrode combination, resulting from this process can then be programmed into the IPG 14. Details regarding the steering of stimulation energy amongst electrodes are further disclosed in U.S. Pat. No. 6,052,624, which is expressly incorporated herein by reference.

In another embodiment, the improved stimulation parameters may be determined by correlating the measured physiological end-functions to a desired performance, and with knowledge of past performance and the operational constraints of the IPG 14, determining the stimulation parameters to be programmed into the IPG 14. For instance, normative data for a physiological end-function may be known in the literature and used as a reference for improving the performance of the patient by adjustment of stimulation parameters as described above. Furthermore, past patient physiological performance profiles may be recorded in a database for the patient and compared to for the adjustment methods. An example of this could be gait performance coupled with energy consumption in which speed of gait, stride length, cadence, and joint excursions coupled with the energy utilized (as measured by oxygen uptake) could be used act as a reference for future stimulation parameter adjustments.

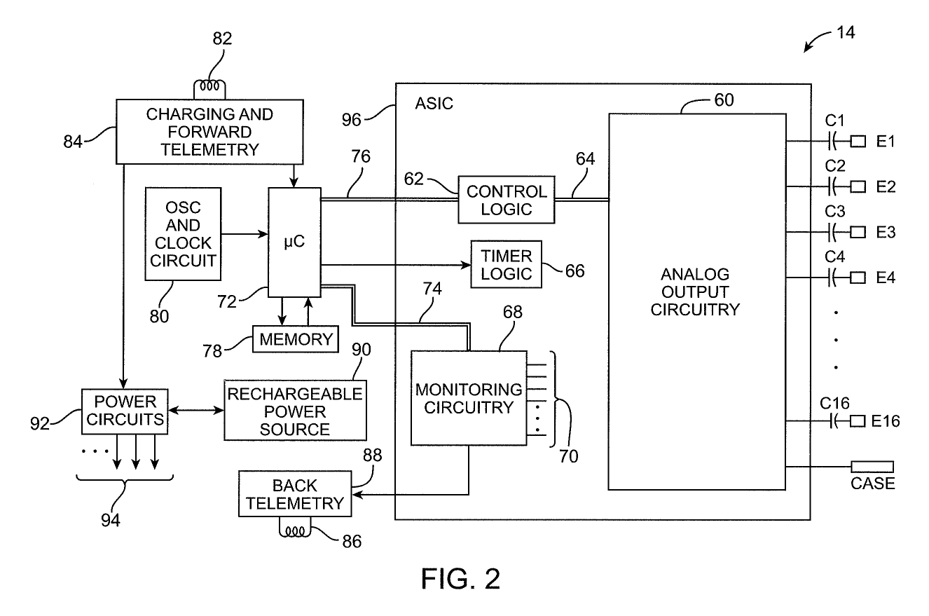

Turning next to FIG. 2, the main internal components of the IPG 14 will now be described. The IPG 14 includes analog output circuitry 60 capable of individually generating electrical stimulation pulses via capacitors C1-C16 at the electrodes 26 (designated E1-E16) of specified amplitude under control of control logic 62 over data bus 64. The duration of the electrical stimulation (i.e., the width of the stimulation pulses), is controlled by the timer logic circuitry 66. The analog output circuitry 60 may either comprise independently controlled current sources for providing stimulation pulses of a specified and known amperage to or from the electrodes 26, or independently controlled voltage sources for providing stimulation pulses of a specified and known voltage at the electrodes 26 or to multiplexed current or voltage sources that are then connected to the electrodes 26. The operation of this analog output circuitry, including alternative embodiments of suitable output circuitry for performing the same function of generating stimulation pulses of a prescribed amplitude and width, is described more fully in U.S. Pat. Nos. 6,516,227 and 6,993,384, which are expressly incorporated herein by reference.

The IPG 14 further comprises monitoring circuitry 68 for monitoring the status of various nodes or other points 70 throughout the IPG 14, e.g., power supply voltages, temperature, battery voltage, and the like. The monitoring circuitry 68 is also configured for measuring electrical parameter data (e.g., electrode impedance and/or electrode field potential). The IPG 14 further comprises processing circuitry in the form of a microcontroller (μC) 72 that controls the control logic over data bus 74, and obtains status data from the monitoring circuitry 68 via data bus 66. The IPG 14 additionally controls the timer logic 56. The IPG 14 further comprises memory 78 and oscillator and clock circuit 80 coupled to the μC 72. The μC 72, in combination with the memory 78 and oscillator and clock circuit 80, thus comprise a microprocessor system that carries out a program function in accordance with a suitable program stored in the memory 78. Alternatively, for some applications, the function provided by the microprocessor system may be carried out by a suitable state machine.

Thus, the μC 72 generates the necessary control and status signals, which allow the μC 72 to control the operation of the IPG 14 in accordance with a selected operating program and stimulation parameters. In controlling the operation of the IPG 14, the μC 72 is able to individually generate stimulus pulses at the electrodes 26 using the analog output circuitry 60, in combination with the control logic 62 and timer logic 66, thereby allowing each electrode 26 to be paired or grouped with other electrodes 26, including the monopolar case electrode, to control the polarity, amplitude, rate, pulse width and channel through which the current stimulus pulses are provided. The μC 72 facilitates the storage of electrical parameter data measured by the monitoring circuitry 68 within memory 78.

The IPG 14 further comprises a receiving coil 82 for receiving programming data (e.g., the operating program and/or stimulation parameters) from the external programmer (i.e., the RC 20 or CP 22) in an appropriate modulated carrier signal, and charging, and circuitry 84 for demodulating the carrier signal it receives through the receiving coil 82 to recover the programming data, which programming data is then stored within the memory 78, or within other memory elements (not shown) distributed throughout the IPG 14.

The IPG 14 further comprises back telemetry circuitry 86 and a transmission coil 88 for sending informational data to the external programmer. The back telemetry features of the IPG 14 also allow its status to be checked. For example, when the external programmer initiates a programming session with the IPG 14, the capacity of the battery is telemetered, so that the external programmer can calculate the estimated time to recharge. Any changes made to the current stimulus parameters are confirmed through back telemetry, thereby assuring that such changes have been correctly received and implemented within the implant system. Moreover, upon interrogation by the external programmer, all programmable settings stored within the IPG 14 may be uploaded to the external programmer.

The IPG 14 further comprises a rechargeable power source 90 and power circuits 92 for providing the operating power to the IPG 14. The rechargeable power source 90 may, e.g., comprise a lithium-ion or lithium-ion polymer battery or other form of rechargeable power. The rechargeable battery 90 provides an unregulated voltage to the power circuits 92. The power circuits 92, in turn, generate the various voltages 94, some of which are regulated and some of which are not, as needed by the various circuits located within the IPG 14. The rechargeable power source 90 is recharged using rectified AC power (or DC power converted from AC power through other means, e.g., efficient AC-to-DC converter circuits, also known as “inverter circuits”) received by the receiving coil 82. To recharge the power source 90, an external charger (not shown), which generates the AC magnetic field, is placed against, or otherwise adjacent, to the patient's skin over the implanted IPG 14. The AC magnetic field emitted by the external charger induces AC currents in the receiving coil 82. The charging and forward telemetry circuitry 84 rectifies the AC current to produce DC current, which is used to charge the power source 90. While the receiving coil 82 is described as being used for both wirelessly receiving communications (e.g., programming and control data) and charging energy from the external device, it should be appreciated that the receiving coil 82 can be arranged as a dedicated charging coil, while another coil, such as coil 88, can be used for bi-directional telemetry.

As shown in FIG. 2, much of the circuitry included within the IPG 14 may be realized on a single application specific integrated circuit (ASIC) 96. This allows the overall size of the IPG 14 to be quite small, and readily housed within a suitable hermetically-sealed case. Alternatively, most of the circuitry included within the IPG 14 may be located on multiple digital and analog dies, as described in U.S. patent application Ser. No. 11/177,503, filed Jul. 8, 2005, which is incorporated herein by reference in its entirety. For example, a processor chip, such as an application specific integrated circuit (ASIC), can be provided to perform the processing functions with on-board software. An analog IC (AIC) can be provided to perform several tasks necessary for the functionality of the IPG 14, including providing power regulation, stimulus output, impedance measurement and monitoring. A digital IC (DigIC) may be provided to function as the primary interface between the processor IC and analog IC by controlling and changing the stimulus levels and sequences of the current output by the stimulation circuitry in the analog IC when prompted by the processor IC.

It should be noted that the diagram of FIG. 2 is functional only, and is not intended to be limiting. Those of skill in the art, given the descriptions presented herein, should be able to readily fashion numerous types of IPG circuits, or equivalent circuits, that carry out the functions indicated and described, which functions include not only producing a stimulus current or voltage on selected groups of electrodes, but also the ability to measure electrical parameter data at an activated or non-activated electrode. Such measurements allow impedance to be determined (used with a first embodiment of the invention) or allow electric field potentials to be measured (used with a second embodiment of the invention), as described in more detail below.

Additional details concerning the above-described and other IPGs may be found in U.S. Pat. No. 6,516,227, U.S. Patent Publication No. 2003/0139781, and U.S. patent application Ser. No. 11/138,632, entitled “Low Power Loss Current Digital-to-Analog Converter Used in an Implantable Pulse Generator,” which are expressly incorporated herein by reference. It should be noted that rather than an IPG, the DBS system 10 may alternatively utilize an implantable receiver-stimulator (not shown) connected to the stimulation leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

The patient monitor 18 may take the form of any one of a variety of monitoring devices, several of which are commercially available. The patient monitor 18 may include a peripheral device that measures the physiological end-function of the patient, and a processor, such as a computer, that quantifies the dysfunction of the patient based on the measured physiological end-function. The processor may be separate from the CP 22 (or RC 20), or a portion or the entirety of the processor may be incorporated into the CP 22 (or RC 20).

For example, the patient monitor 18 may be a quantitative motor assessment system that objectively quantifies dysfunctions that involve muscle spasticity (tremor) or muscle limitations (e.g., bradykinesia or rigidity). Exemplary quantitative motor assessment systems designed specifically for patients suffering from Parkinson's Disease are marketed by CleveMed under the trademarks ParkinSense™ and Kinesia™. The ParkinSense™ and Kinesia™ systems are portable, wireless devices that can be attached to the patient using a ring sensor that is placed on a finger of the patient to perform physiological measurements and a wrist module that is electrically coupled to the wrist module via a cable and provides battery power, memory, and real-time transmission. The ring sensor is capable of performing three-dimensional motion detection (using three gyroscopes to obtain orthogonal angular rates, and three accelerometers to obtain orthogonal accelerations). Additional electrodes electrically coupled to the wrist module may be attached to the patient's skin to detect muscle activity (electromyograms). The resulting physiological data is wirelessly transmitted (using Bluetooth radio communication) from the wrist module to a computer, which quantifies the movement disorder based on the data. The computer has a software interface that provides a database to manage and review recorded data files, and clinical videos to guide the patient or clinician through a motor exam based on the Unified Parkinson's Disease Rating Scale, which results in an objective score.

As another example, the patient monitor 18 may be an isokinetic dynamometer that objectively quantifies dysfunctions that involve neuromuscular torque and power and resulting limb movement. An exemplary isokinetic dynamometer specifically designed for performing neuromuscular testing is marketed by Biodex under the trademark Biodex System 3™, The Biodex System 3™ includes a positioning chair in which the patient can be positioned to perform a variety of physical exercises involving movement of the patient's limbs, and a computer system for controlling and implementing the physical exercises, and quantitatively measuring the patient's neuromuscular ability.

As still another example, the patient monitor 18 may be a balance testing device that objectively quantifies dysfunctions that involve balance. An exemplary balance test device specifically designed for performing balance testing is marketed by Biodex under the trademark Balance System SD™. The Balance System SD™ includes a base on which a patient stands and a computer system with a visual biofeedback display that guides the patient through a variety of balancing tests. The base can be manipulated by the computer system to perform the tests in either a static (base remains stable) or dynamic format (base moves). The computer system displays a variety of biofeedback prompts for performing balancing tests, and quantifies the patient's ability to balance based on the performance of these balancing tests.

As still another example, the patient monitor 18 may be a motion tracking system that objectively quantifies dysfunctions that involve any number of aspects, including posture, balance, motor control, and gait. An exemplary motion tracking system is marketed by Vicon under the trademark Peak Motus™. The Peak Motus™ motion tracking system includes a number of high speed video cameras mounted around a room, a number of reflective markers mounted to various locations on the patients body, and a computer for tracking the motion of the patient's limbs, including joint flexion/extension, based on the detected images of the reflective markers as the patient moves about. Based on the tracked motion, the computer can quantify the posture, balance, motor control, and gait of the patient.

While non-invasive means for measuring physiological end-functions have been described herein, invasive means for measuring physiological end-functions may be used. For example, a goniometer could be implanted within the limbs of a patient to measure joint flexion/extension of the limb. Use of an invasive means, such as a goniometer, is advantageous in that it will allow for continuous measurements (or at least more repeatedly) of the physiological end-functions.

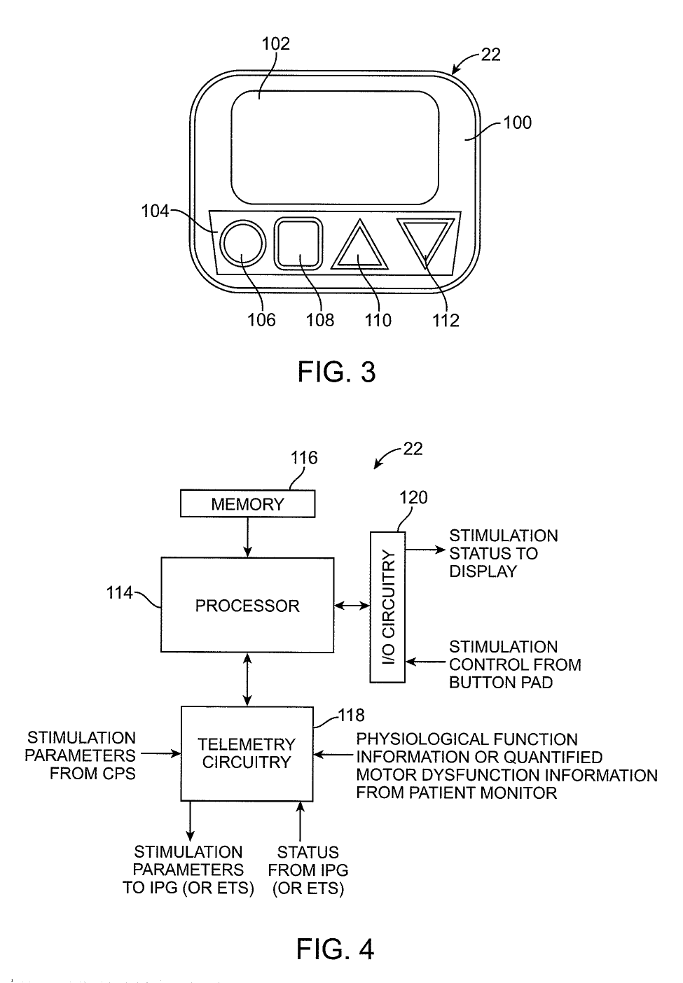

Referring now to FIG. 3, one exemplary embodiment of an RC 20 will now be described. As previously discussed, the RC 20 is capable of communicating with the IPG 14, patient monitor 18, or CP 22. The RC 20 comprises a casing 100, which houses internal componentry (including a printed circuit board (PCB)), and a lighted display screen 102 and a button pad 104 carried by the exterior of the casing 100. In the illustrated embodiment, the display screen 102 is a lighted flat panel display screen, and the button pad 104 comprises a membrane switch with metal domes positioned over a flex circuit, and a keypad connector connected directly to a PCB. The button pad 104 includes a series of buttons 106, 108, 110, and 112, which allow the IPG 22 to be turned ON and OFF, provide for the adjustment or setting of stimulation parameters within the IPG 14, and provide for selection between screens.

In the illustrated embodiment, the button 106 serves as an ON/OFF button that can be actuated to turn the IPG 14 ON and OFF. The button 108 serves as a select button that allows the RC 20 to switch between screen displays and/or parameters. The buttons 110 and 112 serve as up/down buttons that can actuated to increment or decrement any of stimulation parameters of the pulse generated by the IPG 14, including pulse amplitude, pulse width, and pulse rate. For example, the selection button 108 can be actuated to place the RC 16 in an “Pulse Amplitude Adjustment Mode,” during which the pulse amplitude can be adjusted via the up/down buttons 110, 112, a “Pulse Width Adjustment Mode,” during which the pulse width can be adjusted via the up/down buttons 110, 112, and a “Pulse Rate Adjustment Mode,” during which the pulse rate can be adjusted via the up/down buttons 110, 112. Alternatively, dedicated up/down buttons can be provided for each stimulation parameter. Alternatively, rather than using up/down buttons, any other type of actuator, such as a dial, slider bar, or keypad, can be used to increment or decrement the stimulation parameters. Thus, it can be appreciated that any stimulation parameters programmed into the RC 20, and thus, the IPG 14, can be adjusted by the user via operation of the keypad 104. The RC 20 may have another button (not shown) that can be actuated to place the RC 20 either in a manual programming mode or an automatic programming mode, as previously discussed.

Referring to FIG. 4, the internal components of an exemplary RC 20 will now be described. The RC 20 generally includes a processor 114 (e.g., a microcontroller), memory 116 that stores an operating program for execution by the processor 114, as well as stimulation parameters, input/output circuitry, and in particular, telemetry circuitry 118 for outputting stimulation parameters to the IPG 22 and receiving status information from the IPG 14, and input/output circuitry 120 for receiving stimulation control signals from the button pad 104 and transmitting status information to the display screen 102 (shown in FIG. 3). As well as controlling other functions of the RC 20, which will not be described herein for purposes of brevity, the processor 114 generates new stimulation parameters in response to the user operation of the button pad 104. These new stimulation parameters would then be transmitted to the IPG 14 via the telemetry circuitry 118, thereby adjusting the stimulation parameters stored in the IPG 14 and/or programming the IPG 14 with the stimulation parameters. The telemetry circuitry 118 can also be used to receive stimulation parameters from the CP 22 and/or physiological end-function information or quantified dysfunction information from the patient monitor 18. Further details of the functionality and internal componentry of the RC 20 are disclosed in U.S. Pat. No. 6,895,280, which has previously been incorporated herein by reference.

As briefly discussed above, modifying and programming the stimulation parameters in the programmable memory of the IPG 14 after implantation can also be performed by a physician or clinician using the CP 22, which can directly communicate with the IPG 14 or indirectly communicate with the IPG 14 via the RC 16. As shown in FIG. 1, the overall appearance of the CP 22 is that of a laptop personal computer (PC), and in fact, may be implemented using a PC that has been appropriately configured to perform the functions described herein. Thus, the programming methodologies can be performed by executing software instructions contained within the CP 22. Alternatively, such programming methodologies can be performed using firmware or hardware. In any event, the CP 22 determines the improved stimulation parameters based on the measured physiological end-functions or quantified dysfunction information and for subsequently programming the IPG 14 with the optimum or effective stimulation parameters.

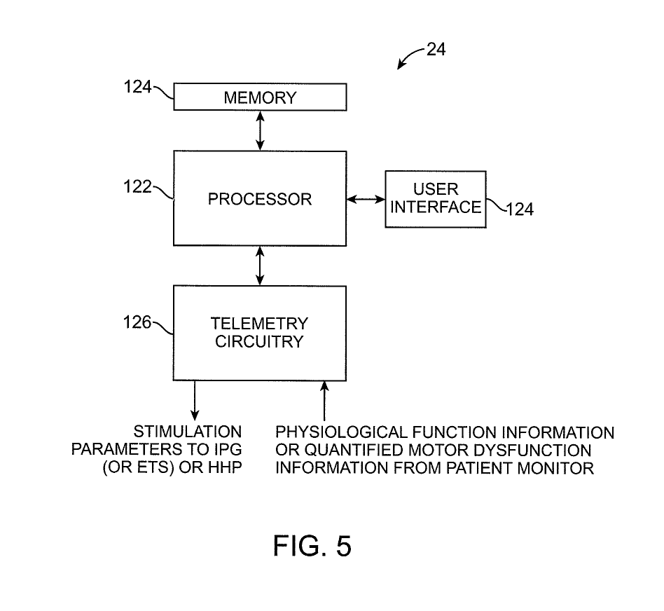

To this end, the functional components of the CP 22 will now be described with reference to FIG. 5. The CP 22 generally includes a processor 122 (e.g., a central processor unit (CPU)), memory 124 for storing software that can be executed by the processor 122 to allow a clinician to selectively adjust stimulation parameters to be programmed into the IPG 14, and when the CP 22 is in the automated mode, automatically determining stimulation parameters to be programmed into the IPG 14 based on the measured physiological end-functions or quantified dysfunction information received from the patient monitor 18. The CP 22 further comprises a standard user interface 124 (e.g., a keyboard, mouse, joystick, display, etc.) to allow a clinician to input information and control the process), and telemetry circuitry 126 for receiving the physiological end-function information or quantified dysfunction information from the patient monitor 18, and outputting stimulation parameters to the IPG 14 for adjustment or programming of the stimulation parameters stored in the IPG 14. Further details discussing CPs are disclosed in U.S. Pat. No. 6,909,917, which is expressly incorporated herein by reference.

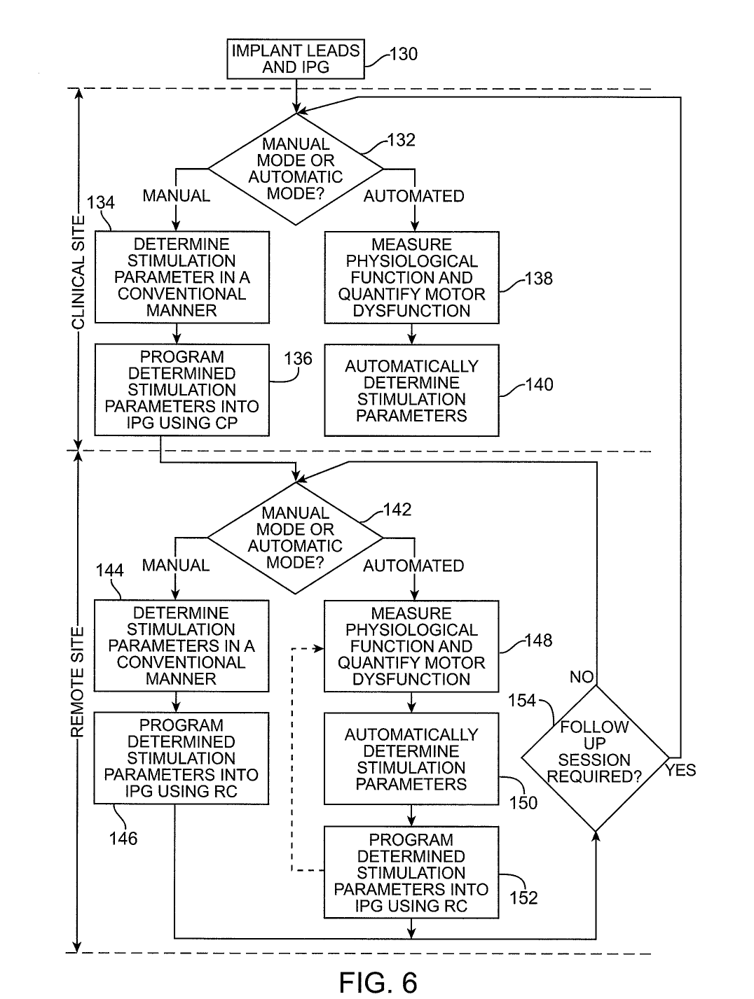

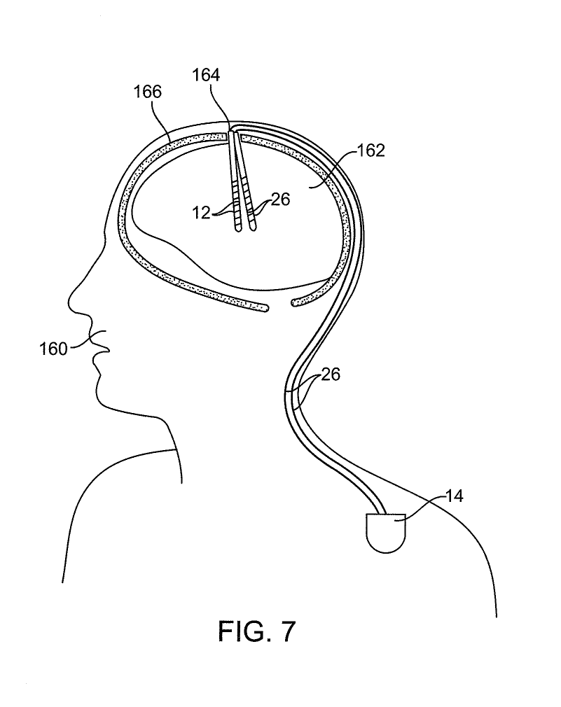

Having described the structure and function of the DBS system 10, its operation will now be described with reference to FIG. 6. First, the stimulation leads 12, the extensions 24 and the IPG 14 are implanted within the patient (step 130). In particular, and with reference to FIG. 7, the stimulation leads 12 are introduced through a burr hole 164 formed in the cranium 166 of a patient 160, and introduced into the parenchyma of the brain 162 of a patient 160 in a conventional manner, such that the electrodes 26 are adjacent a target tissue region whose electrical activity is the source of the dysfunction (e.g., the ventrolateral thalamus, internal segment of globus pallidus, substantia nigra pars reticulate, subthalamic nucleus, or external segment of globus pallidus). Thus, stimulation energy can be conveyed from the electrodes 26 to the target tissue region to change the status of the dysfunction.

The IPG 14 may be generally implanted in a surgically-made pocket in the torso of the patient (e.g., the chest or shoulder region). The IPG 14 may, of course, also be implanted in other locations of the patient's body. The lead extensions 24, which may be subcutaneously advanced underneath the scalp of the patient to the IPG implantation site, facilitates locating the IPG 14 away from the exit point of the stimulation leads 12. In alternative embodiments, the IPG 14 may be directly implanted on or within the cranium 166 of the patient, as described in U.S. Pat. No. 6,920,359, which is expressly incorporated herein by reference. In this case, the lead extensions 24 may not be needed. After implantation, the IPG 14 is used to provide the therapeutic stimulation under control of the patient.

Next, the CP 22 is operated by the clinician to program stimulation parameters within the IPG 14 (steps 132-140). The CP 22 may be operated in either a manual mode or an automated mode (step 132) to program the stimulation parameters within the IPG 14. If the CP 22 is operated in the manual mode, the clinician determines the stimulation parameters to be programmed into the IPG 14 a conventional manner (step 134), and then programs these stimulation parameters into the IPG 14 via the CP 22 (step 136). If the CP 22 is operated in the automated mode, the patient monitor 18 is operated to measure the physiological end-function indicating a change in the status of the dysfunction and optionally quantify the dysfunction based on the measured physiological end-function (step 138), and the CP 22 automatically determines the stimulation parameters (preferably, the optimum or most effective) based on the measured physiological end-function or quantified dysfunction (step 140). In one exemplary method, the CP 22 may be operated in the manual mode to utilize the expert judgment of the clinician as a starting point for determining the stimulation parameters, and then operated in the automated mode to fine-tune the stimulation parameters. The CP 22 may, e.g., automatically determine the stimulation parameters by using the heuristic or correlation approaches discussed above. The CP 22 then programs these stimulation parameters into the IPG 14 without or without the aid of the clinician (i.e., by either automatically programming the IPG 14 with the stimulation parameters or suggesting stimulation parameters to the clinician who can then prompt the RC 14 to program the suggested stimulation parameters into the IPG (step 136).

Once the DBS system 10 is properly fitted to the patient, the stimulation parameters programmed into the IPG 14 may be adjusted at a remote site outside of the clinical setting (steps 142-154). In particular, the RC 20 may optionally be operated between a manual mode and an automated mode (assuming that the patient monitor 18 is ambulatory or otherwise cost efficient to maintain within the patient's home) in a similar manner as the CP 22 (step 142). Notably, it may be necessary to limit the range of effects that could take place during the automated may, which may otherwise require the judgment or intervention of a clinician to oversee full automated operation of the process. If the RC 20 is operated in the manual mode, the patient may determine the stimulation parameters to be programmed into the IPG 14 in a conventional manner (typically, simply by using the RC 20 to adjust the stimulation parameters already programmed into the IPG 14) (step 144), and then may reprogram the adjusted stimulation parameters into the IPG 14 via the RC 20 (step 146). If the RC 20 is operated in the automated mode, the patient monitor 18 is operated to measure the physiological end-function indicating a change in the status of the dysfunction and optionally quantify the dysfunction based on the measured physiological end-function (step 148), the RC 20 automatically determines the stimulation parameters (preferably, the optimum or most effective) based on the measured physiological end-function or quantified dysfunction (step 150), and programs these stimulation parameters into the IPG 14 without or without patient intervention (step 152). Operation of the RC 20 in the automated mode and can be performed continuously (by iteratively performing steps 148-152) to compensate for changes in the dysfunction as a result of disease progression, motor re-learning, etc. If a follow-up programming session is necessary (step 154), steps 132-140 can be repeated.

It should be noted that, while the DBS system 10 and method of using the same has been described in the contact of programming an IPG or other implantable device, an external device, such as an external trial stimulation (ETS) (not shown) may be programmed in the same manner. The major difference between an ETS and the IPG 14 is that the ETS is a non-implantable device that is used on a trial basis after the stimulation leads 12 have been implanted and prior to implantation of the IPG 14, to test the responsiveness of the stimulation that is to be provided. Further details of an exemplary ETS are described in U.S. Pat. No. 6,895,280, which is expressly incorporated herein by reference.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

1. A method of providing therapy to a patient having a dysfunction, comprising:
conveying stimulation energy from a neurostimulator to at least one implanted electrode located within a tissue region of the patient, thereby changing the status of the dysfunction;
measuring a physiological end-function of the patient indicative of the changed status of the dysfunction; and
programming at least one stimulation parameter into the neurostimulator based on the measured physiological end-function.

2. The method of claim 1, wherein the dysfunction is caused by neurological disorder.

3. The method of claim 1, wherein the dysfunction is a motor dysfunction.

4. The method of claim 1, wherein the tissue region is located in the brain.

5. The method of claim 1, wherein the measured physiological end-function is at least one of a kinematic function, an electrical muscle impulse, and a speech pattern.

6. The method of claim 1, wherein the physiological end-function is non-invasively measured.

7. The method of claim 1, wherein the at least one stimulation parameter comprises at least one of a pulse amplitude, pulse width, pulse rate, and electrode combination.

8. The method of claim 1, further comprising conveying stimulation energy from the neurostimulator to the tissue region of the patient in accordance with the at least one stimulation parameter, thereby improving the status of the dysfunction.

9. The method of claim 1, further comprising quantifying the dysfunction based on the measured physiological end-function, wherein the at least one stimulation parameter is programmed into the neurostimulator based on the quantified dysfunction.

10. The method of claim 1, further comprising automatically determining the at least one stimulation parameter in response to the measured physiological end-function.

11. The method of claim 10, wherein the automatic determination of the at least one stimulation parameter is performed heuristically.

12. The method of claim 10, wherein the automatic determination of the at least one stimulation parameter is performed by correlating the measured physiological end-function to a predetermined data set.

13. The method of claim 1, further comprising implanting the neurostimulator into the patient.

14. A neurostimulation system, comprising:
at least one electrical terminal;
output stimulation circuitry configured for outputting stimulation energy to the at least one electrical terminal;
control circuitry configured for controlling the stimulation energy output by the output stimulation circuitry;
monitoring circuitry configured for measuring a physiological end-function of a patient indicative of a changed status of a dysfunction of a patient; and
processing circuitry configured for programming the control circuitry with at least one stimulation parameter based on the measured physiological end-function.

15. The system of claim 14, wherein the dysfunction is a motor dysfunction.

16. The system of claim 14, wherein the measured physiological end-function is at least one of a kinematic function, an electrical muscle impulse, and a speech pattern.

17. The system of claim 14, wherein the monitoring circuitry is configured for non-invasively measuring the physiological end-function.

18. The system of claim 14, wherein the at least one stimulation parameter comprises at least one of a pulse amplitude, pulse width, pulse rate, and electrode combination.

19. The system of claim 14, wherein the processing circuitry is configured for programming the control circuitry with the at least one stimulation parameter to improve the status of the dysfunction when the output stimulation circuitry outputs the stimulation energy to the at least one electrical terminal.

20. The system of claim 14, wherein the monitoring circuitry is configured for quantifying the dysfunction based on the measured physiological end-function, and the processing circuitry is configured for programming the at least one stimulation parameter into the control circuitry based on the quantified dysfunction.

21. The system of claim 14, wherein the processing circuitry is configured for automatically determining the at least one stimulation parameter in response to the measured physiological end-function.

22. The system of claim 21, wherein the processing circuitry is configured for performing the automatic determination of the at least one stimulation parameter heuristically.

23. The system of claim 21, wherein the processing circuitry is configured for performing the automatic determination of the at least one stimulation parameter by correlating the measured physiological end-function to a predetermined data set.

24. The system of claim 14, further comprising telemetry circuitry configured for wirelessly conveying the at least one stimulation parameter from the processing circuitry to the control circuitry.

25. The system of claim 14, further comprising a case containing the at least one electrical terminal, output stimulation circuitry, and control circuitry to form a neurostimulator

26. The system of claim 25, wherein the neurostimulator is implantable.

27. The system of claim 14, wherein the monitoring circuitry and the processing circuitry are contained within one or more computers.

28. An external programmer for a neurostimulator, comprising:
input circuitry configured for receiving information indicative of a changed status of a dysfunction of a patient;
processing circuitry configured for automatically determining at least one programmable stimulation parameter based on the received information; and
output circuitry configured for transmitting the programmable stimulation parameter to the neurostimulator.

29. The programmer of claim 28, wherein the information is a measured physiological end-function.

30. The programmer of claim 29, wherein the measured physiological end-function is at least one of a kinematic function, an electrical muscle impulse, and a speech pattern.

31. The programmer of claim 28, wherein the information is a quantified dysfunction.

32. The programmer of claim 28, wherein the at least one programmable stimulation parameter comprises at least one of a pulse amplitude, pulse width, pulse rate, and electrode combination.

33. The programmer of claim 28, wherein the processing circuitry is configured for defining the at least one programmable stimulation parameter, such that the status of the dysfunction is improved when stimulation energy is delivered to the patient in accordance with the programmable stimulation parameter.

34. The programmer of claim 28, wherein the processing circuitry is configured for performing the automatic determination of the at least one programmable stimulation parameter heuristically.

35. The programmer of claim 28, wherein the processing circuitry is configured for performing the automatic determination of the at least one programmable stimulation parameter by correlating the received information to a predetermined data set.

36. The programmer of claim 28, wherein the output circuitry comprises telemetry circuitry.

37. The programmer of claim 28, wherein the input circuitry, processing circuitry, and output circuitry are contained in a single case.

38-71. (canceled)
